(19) United States
(12) Patent Application Publication
(10) Pub. No.: US 2020/0397344 A1
(43) Pub. Date: Dec. 24, 2020

(54) Upright Posture Device

(71) Applicant: John T. McDonough, JR., Moorhead, MN (US)

(72) Inventor: John T. McDonough, JR., Moorhead, MN (US)

(21) Appl. No.: 16/447,284

(22) Filed: Jun. 20, 2019

(51) Int. Cl. A61B 5/11; A61B 5/00

(52) U.S. Cl. CPC A61B 5/1116; A61B 5/1036; A61B 5/6824; A61B 5/4561

(57) ABSTRACT

An upright posture device for training user's to improve their posture. The upright posture device generally includes a generally straight, bone-shaped rigid alignment element that presses against a user's triceps, providing a reference that can be felt and thus used to align the device with the user's back. The rigid element has two ends and a central portion between each end, the central portion having a first diameter or width. Each end may have a second width that is greater than the first width. The device may also include a strap extending outwardly from each end of the rigid alignment element to form a loop that includes both the rigid element and the strap, and which encircles the user's upper arm. The rigid alignment element may be hollow, and the strap may extend through the rigid alignment element so that it is rotatable about the strap.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND

### Field

Example embodiments in general relate to an upright posture device for helping people improve their posture.

### Related Art

Any discussion of the related art throughout the specification should in no way be considered as an admission that such related art is widely known or forms part of common general knowledge in the field.

Most, if not all, current posture improving devices are attached to, or use, the arms, shoulders, and body to try to correct posture without regard to any input or conscious correction by the user. In other words, such devices attempt to correct posture without conditioning or training users to adjust their own posture as they walk or stand.

### SUMMARY

An example embodiment is directed to an upright posture device. The upright posture device generally includes a generally straight, bone-shaped rigid alignment element that presses against a user's triceps, providing a reference that can be felt and thus used to align the device with the user's back. The rigid element has two ends and a central portion between each end, the central portion having a first diameter or width. Each end may have a second width that is greater than the first width. In a possible embodiment, the central portion may comprise or include a straight, rigid plastic element, and the larger ends may comprise wood or plastic beads, either glued or otherwise attached to, or formed as an integral part of the central portion.

The device may also include a strap extending outwardly from each end of the rigid alignment element to form a loop that includes both the rigid element and the strap, and which encircles the user's upper arm. The rigid alignment element may be hollow, and the strap may extend through the rigid alignment element so that it is rotatable about the strap. In other embodiments, the strap may be pivotally coupled to each end of the rigid alignment element, without extending through it.

The strap may be adapted to wrap around a user's upper arm and to hold the rigid alignment element against the back of the user's upper arm. With the rigid alignment element thus held in place against the user's arm, the device provides for a way for the user to feel where his arms are in relation to his back, and may adjust his posture accordingly, as described more fully below.

In an example embodiment of the upright posture device, the greater width of the two ends prevents the rigid alignment element from rotating about the user's upper arm. In still another possible embodiment, the central portion is cylindrical. Further, the two ends of the rigid alignment element may be substantially spherical. They may not necessarily be perfectly spherical because they may have opening where they connect with or are formed on the central portion, and they may also have open ends to allow for attachment of the strap.

In another example embodiment of the upright posture device, the strap comprises elastic, and it may further comprise two ends that are connectable to each other to form the loop. The two connectable ends comprise complementary hook-and-loop portions. However, in other embodiments, the portion of the strap that is external to the rigid alignment element may be substantially continuous, while still adapted to be rolled up from a user's hand to his upper arm. The strap's elasticity allows for this.

There has thus been outlined, rather broadly, some of the embodiments of the upright posture device in order that the detailed description thereof may be better understood, and in order that the present contribution to the art may be better appreciated. There are additional embodiments of the upright posture device that will be described hereinafter and that will form the subject matter of the claims appended hereto. In this respect, before explaining at least one embodiment of the upright posture device in detail, it is to be understood that the upright posture device is not limited in its application to the details of construction or to the arrangements of the components set forth in the following description or illustrated in the drawings. The upright posture device is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a perspective view of two upright posture devices on a user in accordance with an example embodiment.

FIG. 2 is a perspective view of two upright posture devices in accordance with an example embodiment.

FIG. 3 is another perspective view of two upright posture devices in accordance with an example embodiment.

FIG. 4 is a back view of two upright posture devices on a user in accordance with an example embodiment.

FIG. 5 is a front view of two upright posture devices on a user in accordance with an example embodiment.

FIG. 6 is a top view of two upright posture devices on a user in accordance with an example embodiment.

FIG. 7 is a perspective view of a user putting on an upright posture device accordance with an example embodiment.

FIG. 8 is another perspective view of a user putting on an upright posture device accordance with an example embodiment.

FIG. 9 is a top view of an upright posture device in accordance with an example embodiment.

FIG. 10 is a sectional view of an upright posture device taken at line 10-10 as shown in FIG. 9, in accordance with an example embodiment.

FIG. 11 is a partial perspective view of an upright posture device in accordance with another example embodiment.

FIG. 12 is another perspective view of two upright posture devices in accordance with an example embodiment.

### DETAILED DESCRIPTION

### A. Overview

An example upright posture device 10 has a rigid component that is held in alignment at the back of a user's arm by a flexible or elastic strap 25. Generally, two such devices are used at the same time. Rather than attempting to “force” a user into a correct posture, the posture devices 10 provide a tactile feeling as they press on the user's upper triceps, so that when a user, as instructed, imagines the rigid alignment elements of the devices being aligned with the outermost portion of the user's back, the user will be standing or walking with correct posture. Accordingly, the user becomes trained to develop good posture habits, which can remain even when the user is no longer wearing the devices.

Each upright posture device generally comprises a bone-shaped rigid alignment element 20 having two ends 22 and a central portion 23 between each end, the central portion 23 comprising a first width, wherein each end 22 comprises a second width that is greater than the first width. The device 10 also comprises a strap 25 extending outwardly from each end 22 of the rigid alignment element 20 to form a loop comprising the rigid alignment element 20 and the strap 25. In some example embodiments, the rigid alignment element 20 may be hollow, and the strap 25 may extend through the rigid element 20 such that it is rotatable about the strap 25. In other embodiments, the strap 25 may be pivotally coupled to each end 22 of the rigid alignment element 20.

The loop comprising the strap 25 may be adapted to wrap around a user's upper arm and to hold the rigid alignment element 20 against the back of the user's upper arm. With the rigid alignment element 20 thus held in place against the user's arm, the device 10 provides a way for the user to feel where his arms are in relation to his back, and may adjust his posture accordingly, as described more fully below.

The strap 25 may take several forms. As just one possible example, it may comprise an inner portion 24, which may be elastic and may also be securely fastened or joined to end portions 26. Further, end portions 26 may comprise hook-and-loop fasteners, thus allowing the strap ends to be connected together to form a substantially continuous loop. The loop may be placed over a user's hand and the entire device 10 slid up the user's arm, so that the rigid alignment element 20 may be positioned against the user's triceps, with the strap 25 encircling the upper arm, and the user's bicep.

### B. Rigid Alignment Element

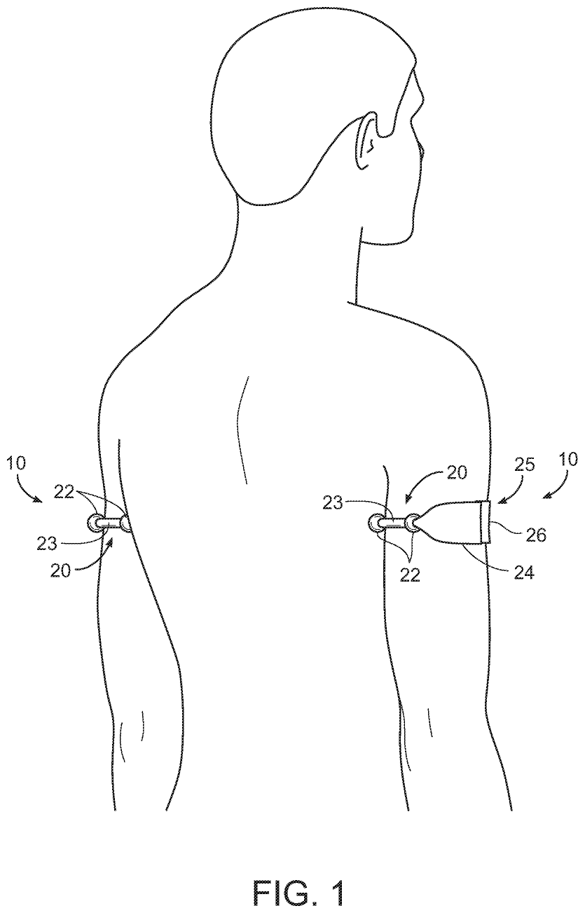

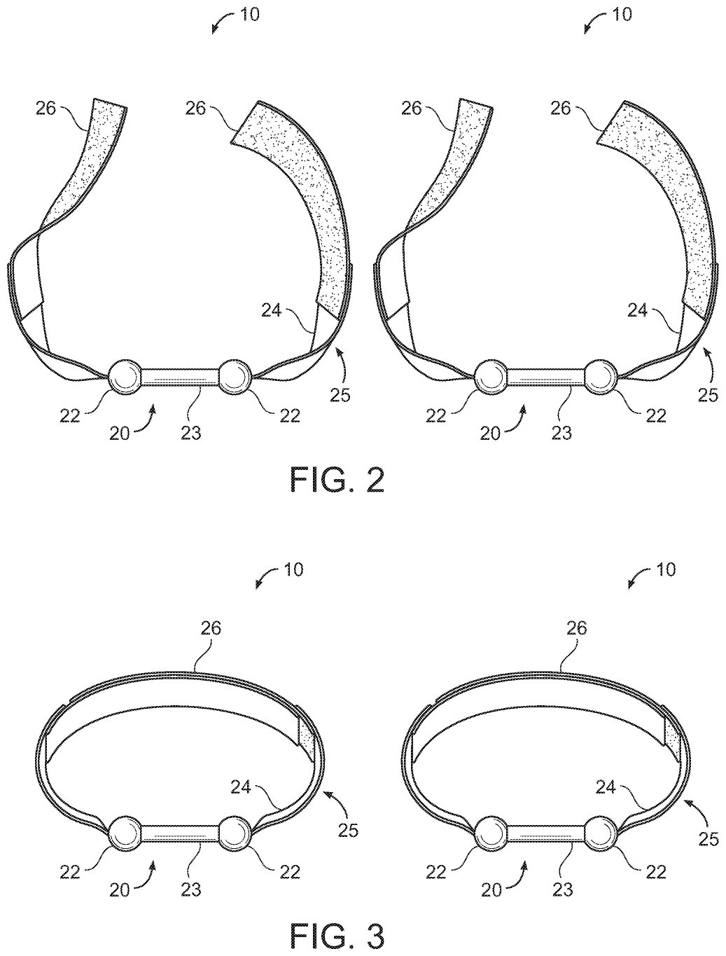

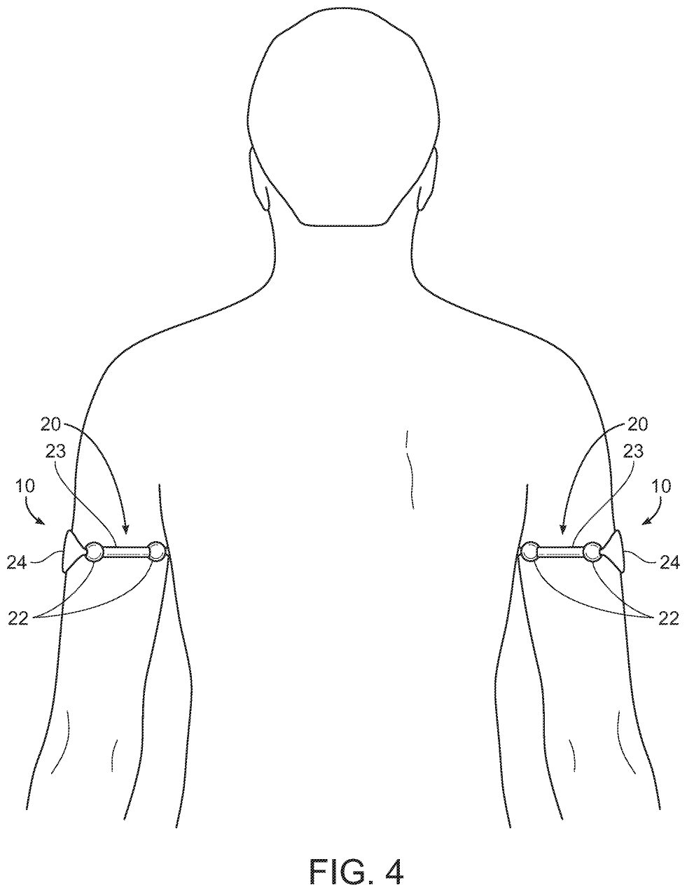

As best shown in FIGS. 2 and 3, the apparatus comprises a rigid alignment element 20 having two ends 22 and a central portion 23 between each end. Because the two ends 22 are larger than the central portion 23, the rigid element 20 will tend to stay in position at the back of the user's arms, especially since the central portion 23 is sized to be about as long as the triceps of a user. Typically, the element 20 will rest on, and press on, a user's upper triceps, as shown for example in FIGS. 1 and 4-6. In a possible embodiment, the central portion 23 may comprise a straight, rigid plastic element, and the larger ends 22 may comprise wood or plastic beads, either glued or otherwise attached to, or formed as an integral part of the central portion 23.

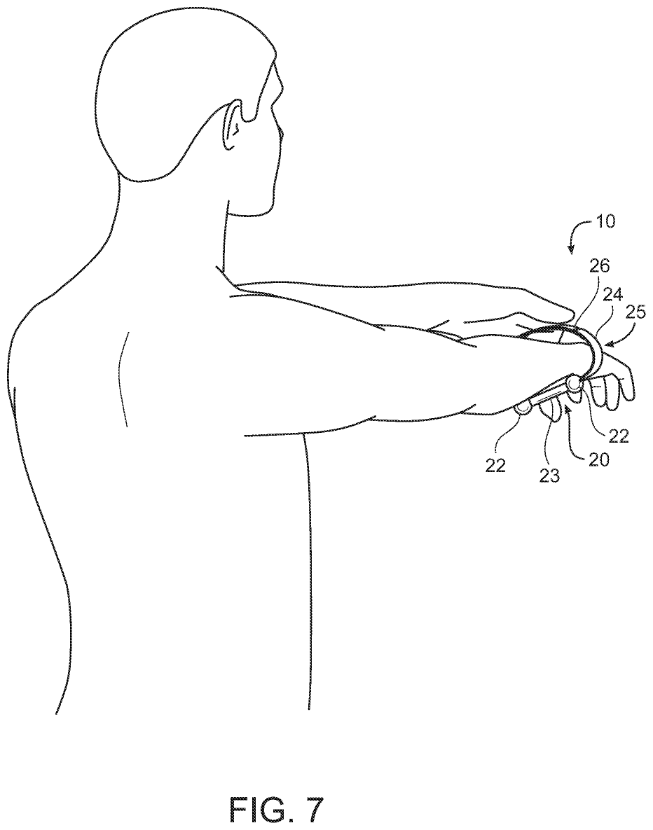

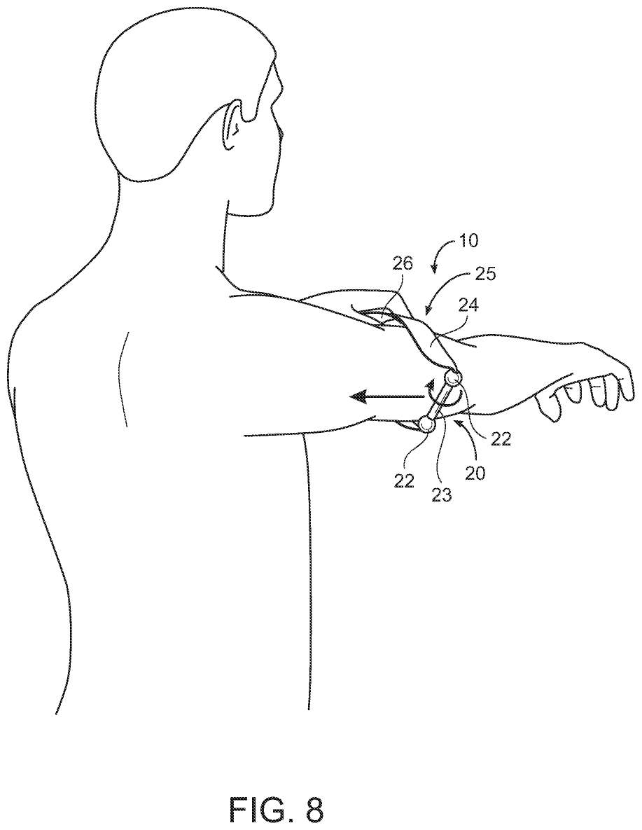

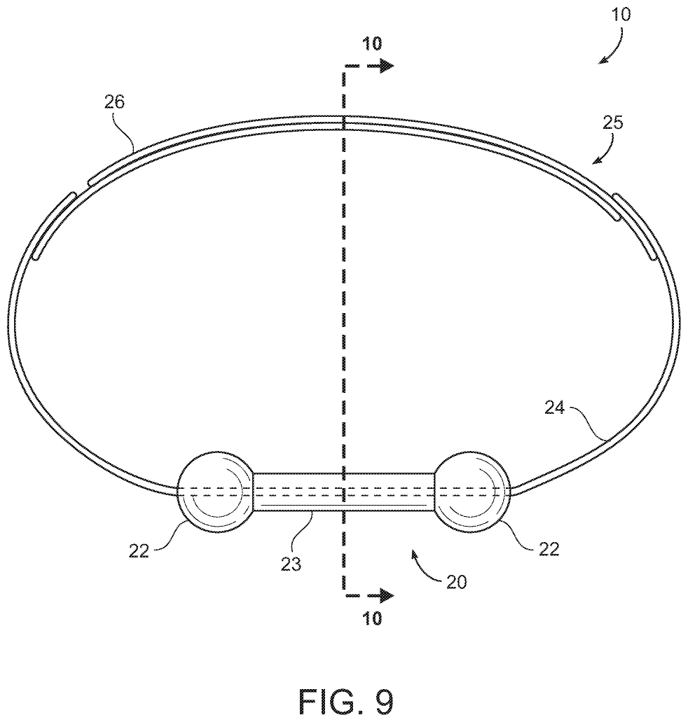

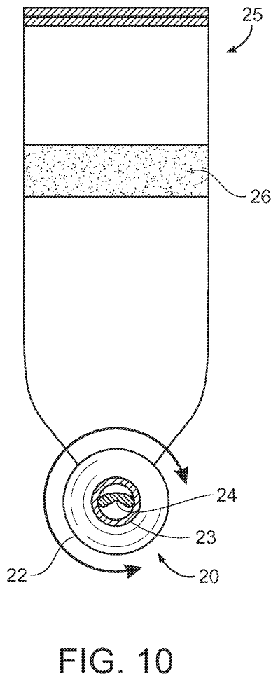

In some embodiments, the rigid alignment element 20 may be hollow, as best shown in FIGS. 9 and 10. In such embodiments, a portion of strap 25 may pass through the rigid alignment element 20. This allows the rigid alignment element 20 to rotate about the strap, which makes the device 10 easier to put on. As shown in FIGS. 7 and 8, if it is adjustable, the strap 25 may be adjusted to an appropriate size before the device is put on. Then, it can be placed over a user's hand as shown in FIG. 7 and pulled farther into position on the user's upper arm, as shown in FIG. 8. As shown in FIGS. 8 and 10, the rigid element 20 can freely rotate as indicated by the arrow, so that the device 10 can be easily put on and removed. Alternatively, embodiments that do not have the ability to rotate are also possible, and that would not detract from the main function of the posture device 10.

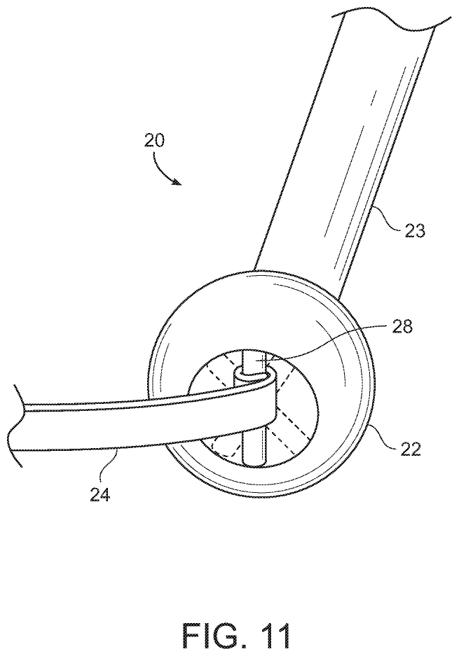

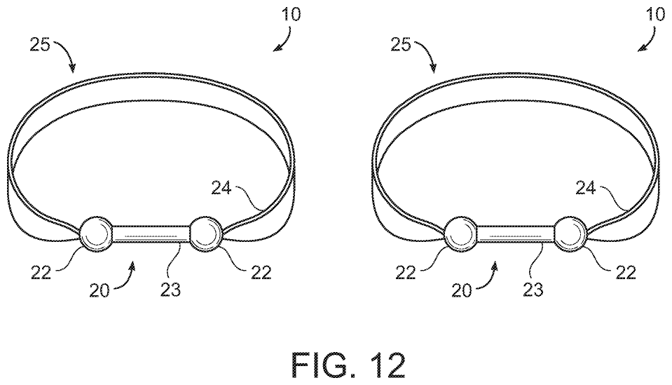

As still another possible alternative, the upright posture device 10 may comprise a rigid alignment element 20 as shown in FIG. 11. In this embodiment, the inner portion 24 of the strap 25 may not necessarily pass all the way through the rigid alignment element 20, but is instead secured to a retention member 28, such as a bar, that can rotate within the enlarged end 22 of rigid alignment element 20. For example, member 28 may be held within end 22 by a bearing or bearings, or it may simply fit within a circular channel (not shown). However this embodiment is implemented, it will function in essentially the same manner as the other illustrated embodiments.

The two ends 22 of the rigid alignment element 20 may be substantially spherical, as shown generally in the Figures. They may not necessarily be perfectly spherical because they may have opening where they connect with or are formed on the central portion 23, and they may also have open ends to allow for attachment of the strap 25. In addition to being generally spherical, the ends 22 may also be made in other shapes, although they will still function as described herein because they have a larger width or diameter than the central portion 23 of the rigid alignment element 20, which helps keep the device 10 in place at the back of a user's upper arm.

As can be seen for example in FIGS. 1 and 4-8, the device 10, and especially the rigid alignment element 20, is small enough to easily fit in a user's pocket, so that two devices 10 can readily be carried for use when needed, and then put away when they are no longer being used.

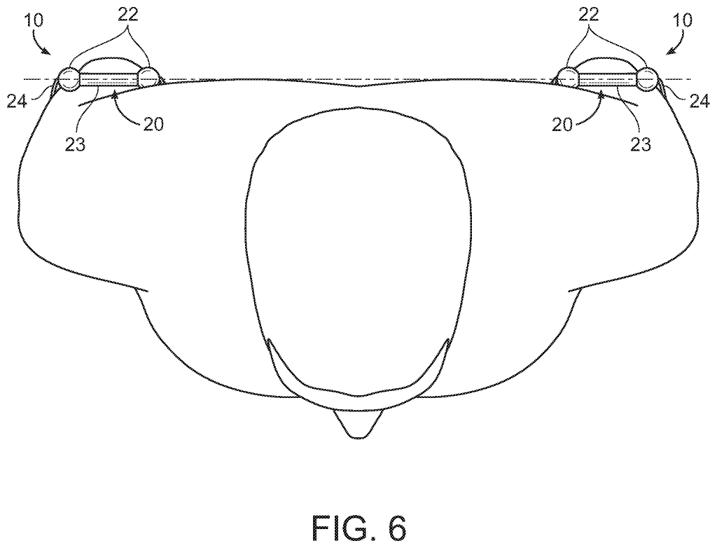

As best shown in FIGS. 1-6, the central portion 23 of rigid alignment element 20 may be cylindrical, and may further have a first width that is smaller than the width or diameter of each end 22. This relationship makes the device tend to remain in position, specifically, aligned along the backs of the user's triceps, as shown in FIGS. 1 and 4-6. Each device 10 will tend to stay in position as best shown in FIG. 6, pressing on the user's upper triceps.

The device 10 also comprises or may comprise a strap extending outwardly from each end 22 of the rigid alignment element 20 to form a loop comprising the rigid alignment element 20 and the strap. In some example embodiments, the rigid alignment element 20 may be hollow, and the strap 25 may extend through the rigid alignment element 20 such that the rigid alignment element 20 is rotatable about the strap 25. In other embodiments, the strap 25 may be pivotally coupled to each end 22 of the rigid alignment element 20, without extending through it.

### C. Strap

The upright posture device 10 also includes a strap 25, which is generally flexible, may be elastic, and serves to hold the rigid alignment element 20 in place so that it presses against a user's triceps. As best shown in FIGS. 2 and 3, the strap 25 of each device may comprise or include an inner portion 24, sewn or connected to an end portion 26, although a one-piece strap is also possible. The inner portion 24 may be elastic, which can help hold the rigid alignment element 20 firmly in place on a user's arm. The end portion 26 of strap 25 may be static (i.e., non-elastic) and may further be, include, or comprise a complementary hook-and-loop fastener or other type of fastener, so that the opposite ends can be adjustably joined or connected together, as shown for example in FIG. 3. The ends may be joined to form a loop that includes the rigid alignment element 20, and the entire device 10 can then be put into use and worn as shown in FIGS. 7, 8, 1, and 4, for example.

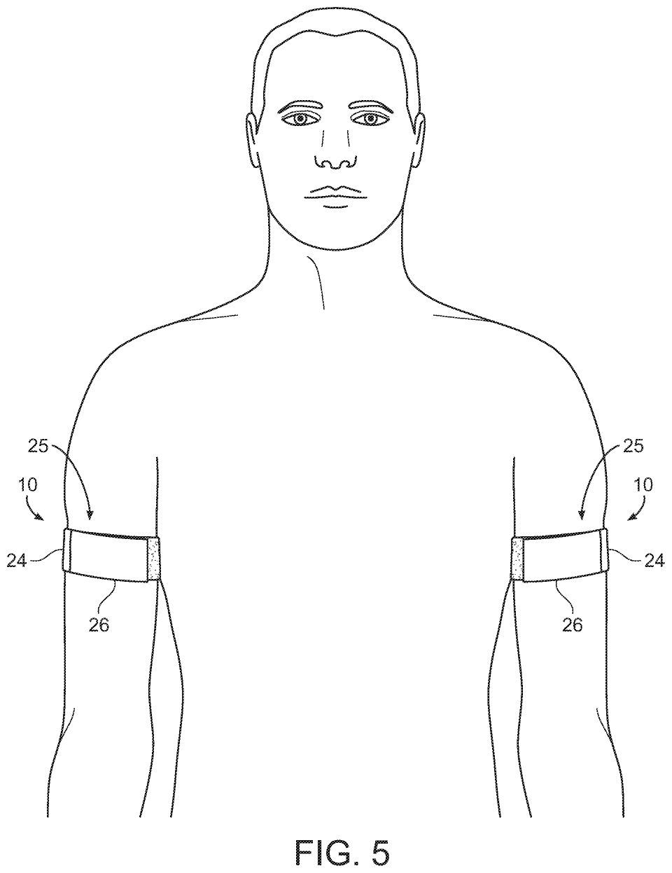

Regardless of what embodiment is used, the inner portion or the entire strap 25 may pass through an opening in the rigid alignment element 20. This creates a simple rotational “attachment” that allows the rigid alignment element 20 to rotate and roll as a user pulls the device into place on the upper arm, as shown in FIGS. 8 and 10. As discussed above, the device 10 may also employ a rotating connection element 28, which may include a bearing or bearings, to accomplish this, as shown in FIG. 11. The strap 25 generally wraps around the front of a user's arms, as best shown in FIG. 5, and may be adjusted to provide a comfortable fit that nevertheless secures the rigid alignment element 20 in place as described herein.

### D. Operation of Preferred Embodiment

In use, the upright posture devices 10 are put on as described above in reference to FIGS. 7 and 8, and worn as shown in FIGS. 1 and 4-6. Generally, two devices are used at any given time, one on each arm, as shown in FIG. 1, for example. The device 10 may include instructions, informing a user to be aware of and align the rigid alignment element 20 with the user's back, imagining, for example, an overall alignment as indicated by the dotted line in FIG. 6. A user may also be instructed to imagine a narrow tube on the outer surface of his back, at the same height as the upright posture devices 10.

When the user's posture is good, the central portion 23 of the rigid alignment element 20 will be aligned with, and have a center line that passes through, the tube. The upright posture devices 10 are generally to be worn during walking or standing, and rather than forcibly moving or changing a user's posture, work by training the user to stand and walk with good posture, by providing for and allowing better awareness of one's posture.

Generally, the devices 10 are only worn during walking or standing, as part of an instructional exercise or routine. The memory of wearing the devices can create good posture by encouraging a user to stand and walk as though the devices are in place even when they are not. Further, because of their extreme portability, in contrast to other devices and techniques, the devices 10 can be readily carried in a user's pockets, and put back on quickly and easily to further encourage good posture habits.

As mentioned and shown, the devices 10 may have adjustable straps 25, that can be quickly and easily adjusted for a perfect, comfortable fit on a user's arms, as shown for example in FIGS. 1 and 4-6. Once adjusted, the straps 25 will generally stay adjusted at the same length, thus forming a consistent loop around the user's arms. However, it can also be appreciated that the devices 10 can be readjusted if necessary, either for the original user or for another person who wishes to use them.

Further, in another embodiment, such as the one shown in FIG. 12, the devices may comprise a strap that has a fixed external length, creating a single loop size. This embodiment can be made by using a single-length strap 25, with its ends permanently or adjustably connected so that the portion of the strap that is outside of rigid alignment element 20 is a smooth, single piece. The strap 25 in such an embodiment may have ends joined inside the central portion 23, or rotatably connected inside the ends 22 as shown in FIG. 11.

Unless otherwise defined, all technical and scientific terms used herein have the same meaning as commonly understood by one of ordinary skill in the art to which this invention belongs. Although methods and materials similar to or equivalent to those described herein can be used in the practice or testing of the upright posture device, suitable methods and materials are described above. All publications, patent applications, patents, and other references mentioned herein are incorporated by reference in their entirety to the extent allowed by applicable law and regulations. The upright posture device may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. An upright posture device, comprising:
a rigid alignment element having two ends and a central portion between each end, the central portion comprising a first width, wherein each end comprises a second width that is greater than the first width; and
a strap extending outwardly from each end of the rigid alignment element to form a loop comprising the rigid alignment element and the strap.

2. The upright posture device of claim 1, wherein the central portion is cylindrical.

3. The upright posture device of claim 2, wherein the two ends are substantially spherical.

4. The upright posture device of claim 1, wherein the wherein the two ends are substantially spherical.

5. The upright posture device of claim 1, wherein the strap comprises elastic.

6. The upright posture device of claim 5, wherein the strap comprises two ends that are connectable to each other to form the loop.

7. The upright posture device of claim 6, wherein the two connectable ends comprise complementary hook-and-loop portions.

8. The upright posture device of claim 1, wherein the strap comprises two ends that are connectable to each other.

9. The upright posture device of claim 8, wherein the two connectable ends comprise complementary hook-and-loop portions.

10. The upright posture device of claim 1, wherein the rigid alignment element is rotatable about the strap.

11. The upright posture device of claim 1, wherein the rigid alignment element is hollow, and wherein the strap extends through the rigid alignment element such that the rigid alignment element is rotatable about the strap.

12. The upright posture device of claim 1, wherein the strap is pivotally coupled to each end of the rigid alignment element.

13. The upright posture device of claim 1, wherein the strap is adapted to wrap around a user's upper arm and to hold the rigid alignment element against the back of the user's upper arm.

14. The upright posture device of claim 13, wherein the greater width of the two ends prevent the rigid alignment element from rotating about the user's upper arm.

15. The upright posture device of claim 14, wherein the rigid alignment element is rotatable about the strap.

16. The upright posture device of claim 14, wherein the strap comprises two ends that are connectable to each other.

17. The upright posture device of claim 16, wherein the two connectable ends comprise complementary hook-and-loop portions.

18. An upright posture device, comprising:
a rigid alignment element having two ends and a central portion between each end, the central portion comprising a first width, wherein each end comprises a second width that is greater than the first width; and
means for holding the rigid alignment element against a user's upper arm.

19. The upright posture device of claim 18, wherein the central portion is cylindrical.

20. The upright posture device of claim 19, wherein the two ends are substantially spherical.
